(19) United States
(12) Patent Application Publication
(10) Pub. No.: US 2008/0208180 A1
(43) Pub. Date: Aug. 28, 2008

(54) ENDOVASCULAR TREATMENT SHEATH HAVING A HEAT INSULATIVE TIP AND METHOD FOR USING THE SAME

(76) Inventors: William A. Cartier, Hampton, NY (US); William M. Appling, Granville, NY (US)

Correspondence Address: AFS / ANGIODYNAMICS, 666 THIRD AVENUE, FLOOR 10, NEW YORK, NY, 10017, US

(21) Appl. No.: 12/109,835

(22) Filed: Apr. 25, 2008

Related U.S. Application Data
(63) Continuation-in-part of application No. 11/777,198, filed on Jul. 12, 2007.
Application No. 11/777,198 is a continuation of application No. 10/613,395, filed on Jul. 3, 2003, now Pat. No. 7,273,478.
Application No. 10/613,395 is a continuation-in-part of application No. 10/836,084, filed on Apr. 30, 2004.
Application No. 10/836,084 is a continuation-in-part of application No. 11/362,239, filed on Feb. 24, 2006.
Application No. 11/362,239 is a continuation of application No. 10/316,545, filed on Dec. 11, 2002, now Pat. No. 7,033,347.
(60) Provisional application No. 60/395,218, filed on Jul. 10, 2002.
(60) Provisional application No. 60/516,156, filed on Oct. 31, 2003.
(60) Provisional application No. 60/914,240, filed on Apr. 26, 2007.

(51) Int. Cl. A61B 18/22

(52) U.S. Cl. 606/15

(57) ABSTRACT

A treatment sheath for use with an energy delivery device, such as an optical fiber, is provided with a heat insulative tip. The treatment sheath includes a longitudinal shaft which is designed to receive the optical fiber, and is inserted into a blood vessel to treat diseases such as varicose veins. During treatment, the energy emitting face of the optical fiber is positioned inside the heat insulative tip of the treatment sheath. The heat insulative tip protects the optical fiber emitting face during the delivery of laser energy and prevents the emitting face from inadvertently contacting the inner vessel wall.

### CROSS REFERENCE TO RELATED APPLICATIONS

This application is a continuation-in-part of U.S. application Ser. No. 11/777,198, filed Jul. 12, 2007, which is a continuation of U.S. application Ser. No. 10/613,395, filed Jul. 3, 2003, now U.S. Pat. No. 7,273,478, which claims priority under 35 U.S.C. Section 119(e) to U.S. Provisional Application Ser. No. 60/395,218 filed Jul. 10, 2002, all of which are incorporated herein by reference.

This application is also a continuation-in-part of U.S. application Ser. No. 10/836,084, filed Apr. 30, 2004, which claims priority under 35 U.S.C. Section 119(e) to U.S. Provisional Application Ser. No. 60/516,156 filed Oct. 31, 2003, all of which are incorporated herein by reference.

This application is also a continuation-in-part of U.S. application Ser. No. 11/362,239, filed Feb. 24, 2006, which is a continuation of U.S. application Ser. No. 10/316,545, filed Dec. 11, 2002, now U.S. Pat. No. 7,033,347, all of which are incorporated herein by reference.

This application also claims priority under 35 U.S.C. Section 119(e) to U.S. Provisional Application Ser. No. 60/914,240, filed Apr. 26, 2007, which is incorporated herein by reference.

### FIELD OF THE INVENTION

The present invention relates to a medical device and method for treatment of blood vessels. More particularly, the present invention relates to an endovascular thermal treatment sheath for treating blood vessels such as varicose veins and method for using the same.

### BACKGROUND OF THE INVENTION

Veins can be broadly divided into three categories: the deep veins, which are the primary conduit for blood return to the heart; the superficial veins, which parallel the deep veins and function as a channel for blood passing from superficial structures to the deep system; and topical or cutaneous veins, which carry blood from the end organs (e.g., skin) to the superficial system. Veins have thin walls and contain one-way valves that control blood flow. Normally, the valves open to allow blood to flow into the deep veins and close to prevent back-flow into the superficial veins. When the valves are malfunctioning or only partially functioning, however, they no longer prevent the back-flow of blood into the superficial veins. This condition is called reflux. As a result of reflux, venous pressure builds within the superficial system. This pressure is transmitted to topical veins, which, because the veins are thin walled and not able to withstand the increased pressure, become dilated, tortuous or engorged.

In particular, venous reflux in the lower extremities is one of the most common medical conditions of the adult population. It is estimated that venous reflux disease affects approximately 25% of adult females and 10% of adult males. Symptoms of reflux include varicose veins and other cosmetic deformities, as well as aching and swelling of the legs. Varicose veins are common in the superficial veins of the legs, which are subject to high pressure when standing. Aside from being cosmetically undesirable, varicose veins are often painful, especially when standing or walking. If left untreated, venous reflux may cause severe medical complications such as bleeding, phlebitis, ulcerations, thrombi and lipodermatosclerosis (LDS). When veins become enlarged, the leaflets of the valves no longer meet properly. Blood collects in the superficial veins, which become even more enlarged. Since most of the blood in the legs is returned by the deep veins, and the superficial veins only return about 10%, they can be removed without serious harm. Non-surgical treatments of the superficial veins may include elastic stockings or elevating the diseased legs. However, while providing temporary relief of symptoms, these techniques do not correct the underlying cause, that is, the faulty valves. Permanent treatments include surgical excision of the diseased segments, ambulatory phlebectomy, and occlusion of the vein through chemical or thermal means, or vein stripping to remove the affected veins.

Surgical excision requires general anesthesia and a long recovery period. Even with its high clinical success rate, surgical excision is rapidly becoming an outmoded technique due to the high costs of treatment and complication risks from surgery. Ambulatory phlebectomy involves avulsion of the varicose vein segment using multiple stab incisions through the skin. The procedure is done on an outpatient basis, but is still relatively expensive due to the length of time required to perform the procedure.

Chemical occlusion, also known as sclerotherapy, is an in-office procedure involving the injection of an irritant chemical into the vein. The chemical acts upon the inner lining of the vein walls causing them to occlude and block blood flow. Although a popular treatment option, severe complications can result, such as skin ulceration, anaphylactic reactions and permanent skin staining. Treatment is limited to veins of a particular size range. In addition, there is a relatively high recurrence rate due to vessel recanalization.

Endovascular thermal therapy is an alternative surgical treatment that is less invasive compared to other surgical treatments and may be used to treat venous reflux diseases. This technique involves delivering thermal energy generated by laser, radio or microwave frequencies to causing vessel ablation or occlusion. Typically a sheath, fiber or other delivery system is percutaneously inserted into the lumen of the diseased vein. Thermal energy is then delivered to the vein wall or blood (depending on the device) as the energy source is withdrawn from the diseased vein.

A treatment sheath is placed into the great saphenous vein, the large subcutaneous superficial vein of the leg and thigh, at a distal location. The sheath is then advanced to within a few centimeters of the point at which the great saphenous vein enters the deep vein system, the sapheno-femoral junction. Typically, a physician will measure the distance from the insertion or access site to the sapheno-femoral junction on the surface of the patient's skin. This measurement is then transferred to the treatment sheath using tape, a marker or some other visual indicator to identify the insertion distance on the sheath shaft. Other superficial veins may also be accessed depending on the origin of reflux.

The treatment sheath is placed using either ultrasonic guidance or fluoroscopic imaging. The physician inserts the sheath into the vein using a visual mark on the sheath as an approximate insertion distance indicator. Ultrasonic or fluoroscopic imaging is then used to guide final placement of the tip relative to the junction. Positioning of the sheath tip relative to the sapheno-femoral junction or other reflux point is very important to the procedure because the sheath tip position is used to confirm correct positioning of the fiber when it is inserted and advanced. Current sheath tips are often difficult to clearly visualize under ultrasonic guidance.

Once the treatment sheath is properly positioned, a flexible optical fiber is inserted into the lumen of the sheath and advanced until the fiber tip extends distally beyond the sheath tip. The laser generator is then activated causing laser energy to be emitted from the distal end of the optical fiber. The energy reacts with the blood in the vessel and causes the blood to boil, thereby producing hot steam bubbles. The gas bubbles transfer thermal energy to the vein wall, causing damage to the endothelium and eventual vein collapse. While the laser remains turned on, the sheath and optical fiber are slowly withdrawn until the entire diseased segment of the vessel has been treated.

Currently available sheaths for endovascular laser treatment of reflux have several drawbacks. Prior art sheaths are designed such that the distal end portion of the fiber extends by approximately 1 cm beyond the distal end of the treatment sheath. Extension beyond the distal end of the sheath is necessary in order to avoid overheating of the polymer sheath tip by the laser energy, which may result in melting and other damage. Ensuring a sufficient distance between the fiber tip and sheath tip avoids any chance of overheating. While extending the energy emitting portion of the fiber beyond the distal end of the sheath avoids overheating, it leaves the fragile fiber tip unprotected and exposed within the vein. The exposed optical fiber tip is often damaged during the procedure as it is being withdrawn through the vein. Blood build up and charring on the energy-emitting face of the fiber tip often results in compromised energy delivery and tip degradation due to intensive heat. A degraded tip will often break leaving unwanted fragments of the optical fiber tip behind in a patient's body after treatment.

In addition to damage to the exposed laser emitting face of the optical fiber tip, a fiber that extends past the sheath tip may inadvertently come into contact with the vessel wall. Even unintended and unwanted contact between the optical fiber tip and the inner wall of the vessel can result in vessel perforation and extravasation of blood into the perivascular tissue. This problem is documented in numerous scientific articles including “Endovenous Treatment of the Greater Saphenous Vein with a 940-nm Diode Laser: Thrombotic Occlusion After Endoluminal Thermal Damage By Laser-Generated Steam Bubble” by T. M. Proebstle, Md., in J of Vasc. Surg., Vol. 35, pp. 729-736 (2002), and “Thermal Damage of the Inner Vein Wall During Endovenous Laser Treatment: Key Role of Energy Absorption by Intravascular Blood” by T. M. Proebstle, Md., in Dermatol. Surg., Vol. 28, pp. 596-600 (2002), both of which are incorporated herein by reference.

When the fiber inadvertently contacts the vessel wall during treatment, intense direct laser energy is delivered to the vessel wall rather than indirect thermal energy created as the blood is converted into gas bubbles. Laser energy in direct contact with the vessel wall can cause the vein to perforate at the contact point and surrounding area. Blood escapes through these perforations into the perivascular tissue, resulting in post-treatment bruising and associated discomfort.

Another problem with currently available sheaths is the difficulty in visualizing the distal end of the exposed fiber, which is very important in correctly positioning the treatment device. Although the sheath may be designed to be ultrasonically visible, it is often difficult for a physician to know where the tip of the optical fiber is in relation to the edge of the sheath. Incorrect placement may result in either incomplete occlusion of the vein or non-targeted thermal energy delivery to the deep femoral vein. Energy that is unintentionally directed into the deep venous system may result in deep vein thrombosis (DVT) and its associated complications including pulmonary embolism (PE).

Therefore, it is desirable to provide an endovascular treatment device and method which protects the energy delivery portion of the energy delivery device from even inadvertent direct contact with the inner wall of the vessel during the emission of energy to ensure consistent thermal heating across the entire vessel circumference, thus avoiding vessel perforation, incomplete vessel collapse, and damage to the optical fiber tip.

### SUMMARY OF THE DISCLOSURE

According to the principles of the present invention, an endovascular treatment sheath for use with an energy delivery device, such as an optical fiber, is provided. The sheath is designed to be inserted into a blood vessel and includes a longitudinal shaft lumen for receiving the optical fiber. The distal end of the sheath includes a heat insulative tip, which protects the optical fiber tip during the delivery of laser energy through the optical fiber.

In one aspect of the invention, the heat insulative tip may be made of ceramic material, which enables the tip to be heat resistant and echogenic. In another aspect of the invention, the heat insulative tip may be made of a glass material. In yet another aspect of the invention, the insulating tip may be made of a high-temperature resistant polymer.

The heat insulative tip of the present treatment sheath surrounds and protects the energy emitting face of the optical fiber and prevents the light emitting face from inadvertently contacting the inner wall of the vessel, thereby preventing vessel perforation and extravasation of blood into the perivascular tissue.

### BRIEF DESCRIPTION OF THE DRAWINGS

FIG. 1A is a plan view of a treatment sheath of the present invention with the insulating tip.

FIG. 1B is a plan view of the optical fiber with fiber connector of the present invention.

FIG. 2 is a plan view of the optical fiber assembled with the treatment sheath of the present invention.

FIG. 3 is a cross-sectional view of the distal section of the sheath enclosing the distal section of the optical fiber of the present invention.

FIG. 4 is a perspective view of the insulating tip component of the present invention.

FIG. 5 is an end view of the distal end of the insulating tip of the present invention.

FIG. 6A is a partial cross-sectional view of an additional embodiment of the distal end of the treatment sheath enclosing the optical fiber of the present invention.

FIG. 6B is a partial cross-sectional view of an additional embodiment of the distal end of the treatment sheath enclosing the optical fiber of the present invention.

FIGS. 7A through 7C is a series of plan views of the treatment sheath device within the vein depicting the method of the present invention.

FIG. 8 is a flowchart depicting the method steps for performing endovascular thermal treatment using the device of FIG. 2.

### DETAILED DESCRIPTION OF THE INVENTION

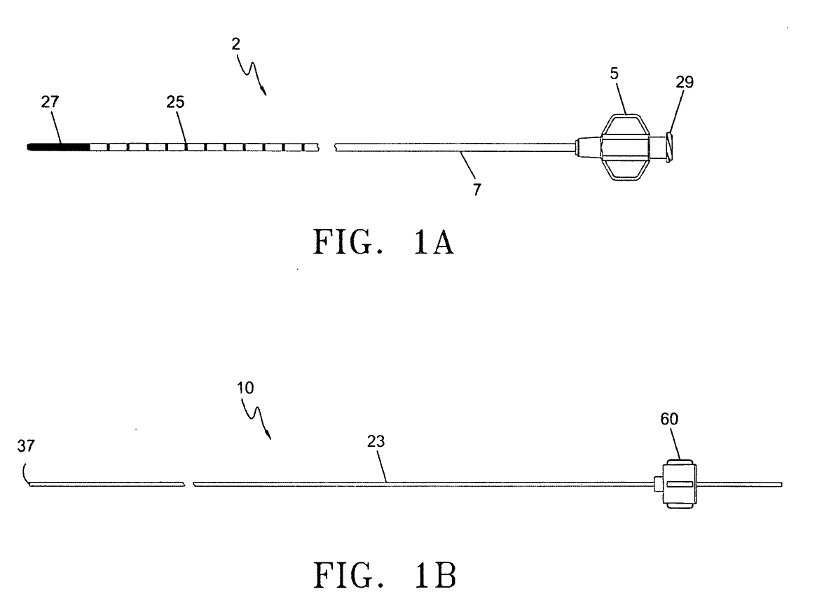

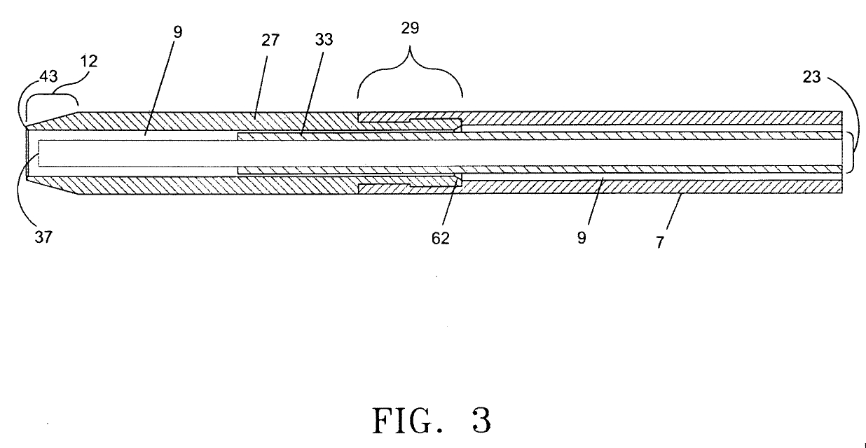

The present invention is illustrated in FIGS. 1 through 8. A treatment sheath 2 is illustrated in FIG. 1A. The treatment sheath 2 includes a proximal sheath hub 5, a sheath shaft 7 and an insulating tip 27 at the distal end. Extending from the hub 5 to the insulating tip 27 is a through lumen 9 (see FIG. 3). The sheath shaft may optionally include depth/distance markers 25. The sheath shaft 7 may also optionally include a reinforcement metallic element embedded within the polymer shaft 7 such as that disclosed in U.S. patent application Ser. No. 10/836,084, which is incorporated herein by reference. The hub 5 includes a standard luer threaded proximal end (treatment sheath connector) 29 for connection to an optical fiber connector 60 (FIG. 1B) or other interventional devices. Hub fittings other than those specifically described herein are within the scope of this invention.

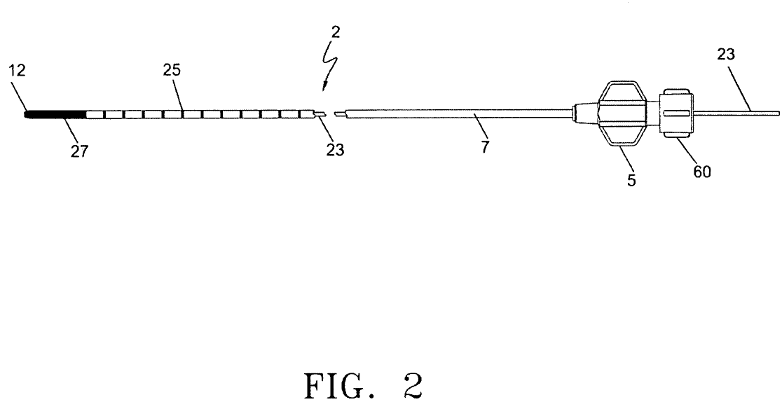

FIG. 1B illustrates the energy delivery device of the current invention. In particular, the energy delivery device shown is an optical fiber using laser as thermal energy. The optical fiber 10 includes a fiber shaft 23, an energy-emitting distal face 37, and a fiber connector 60. The fiber connector 60 is attached to fiber shaft 23, such as that disclosed in U.S. patent application Ser. No. 11/362,239, which is incorporated herein by reference. The fiber connector 60 is bonded to the fiber shaft 23 at a predetermined distance from the fiber energy emitting tip 37, so that when coupled with the sheath hub 5, as shown assembled in FIG. 2, the distal end 37 of the optical fiber 10 is positioned completely within and protected by the sheath insulating tip 27. Although luer threaded hubs are typically used in the medical device industry, any mating connection for locking two medical components together may be used.

FIG. 2 illustrates the sheath 2 and fiber 10 of the present invention in an assembled state. The optical fiber shaft 23 is coaxially arranged within the longitudinal lumen 9 of the sheath 2. As illustrated in FIG. 3, the fiber shaft 23 includes a jacket 33 which provides protection to the cladding and core of the fiber shaft 23. The fiber shaft 23 is stripped of its jacket 33 at the distal end for approximately 0.180 inches to expose the cladding and core. The light emitting face 37 of the optical fiber shaft 23 is ground and polished to form a flat face. The light emitting face 37 of the optical fiber 23 tip directs laser energy forward in a longitudinal direction. The light emitting face 37 of the optical fiber 23 is positioned within the insulating tip 27 of sheath 2. The front face 43 of the insulating tip 27 extends distally beyond the light emitting face 37 of the optical fiber 23 and completely surrounds and protects the optical fiber 23. The length between the outer face 43 of the insulating sheath tip 27 and the light emitting face 37 of the optical fiber 23 is approximately 0.012 inches.

The treatment sheath 2 is a tubular structure that is preferably composed of a flexible, low-friction material such as nylon. Endovenous treatment sheaths are typically 45 centimeters in length, although 60 and 65 centimeter sheaths are also well known in the art. The sheath 2 typically has an outer diameter of 0.079 inches and an inner diameter of about 0.055 inches, although other diameters can be used for different optical fiber sizes. The insulating tip 27 is located at the distal portion of the sheath 2. The insulating tip 27 can have a tapered outer profile. Preferably, only the distal end of the insulating tip 27 has a tapered outer profile. As is well known in the art, the taper provides a smooth transition from the outer diameter of the insulating tip 27, approximately 0.079 inches, to the smaller outer diameter of the insulating tip 27 front face 43, approximately 0.055 inches. The taper aids in insertion and advancement of the sheath 2. The tapered tip section 12 may be as short or as long as practical in order to ensure ease of entry and advancement. Optimally, the tapered tip section 12 is approximately 0.137 inches in length (3.5 mm), but may range from 0 to 5 mm in length. The angle of the tapered tip may be approximately 5 degrees relative to the longitudinal axis of the sheath, but any suitable angle may be also be used.

As shown in FIG. 3, the sheath tip 27 has an inner diameter of 0.045 inches for substantially the entire length of the component. The insulating tip 27 inner diameter of 0.045 inches is designed to provide a centering function for the fiber so as to more accurately position the energy-emitting fiber face 37 to deliver laser energy in a forward longitudinal direction during treatment. The fiber shaft 23 has a diameter of approximately 0.041 inches, which includes the cladding and the jacket 33. The fiber shaft 23 is coaxially arranged within the lumen 9 of the insulating tip 27 with approximately 0.002 inches of annular space (with jacket 33). The inner diameter of the heat insulative tip 27 is smaller than the inner diameter of the sheath shaft 7. This arrangement allows the fiber shaft 23 to be easily advanced in a forward direction and, once the fiber connector 60 is locked to the sheath hub 5, the fiber shaft 23 is maintained in a general centering position within the insulating tip 27 lumen 9.

As further shown in FIG. 3, the inner diameter of insulating tip 27 tapers outwardly near the proximal end to form internal chamfer 62. Internal chamfer 62 transitions to an enlarged inner diameter of 0.055 inches, to match the internal diameter of the sheath shaft 7. The chamfer 62 provides a gradually inwardly tapering ramp to facilitate advancement of the fiber shaft 23 into the insulating tip 27 portion of the sheath 2.

The insulating tip 27 provides a protective barrier between the vein wall and the light emitting face 37 of the optical fiber shaft 23 during endovenous laser treatment of a vessel, such that the light emitting face 37 of the fiber shaft 23 is never directly exposed to the vessel wall, thereby minimizing perforations of the vessel. The protective function of the insulating tip 27 also minimizes accumulation of blood on the fiber face 37, which is known to cause charring and increased temperatures at the distal region of the fiber. The insulating tip 27 is composed of a high temperature-resistant material which ensures that the distal end portion of the sheath shaft 7 does not degrade under the elevated temperatures. By protecting the fragile fiber tip 37 from the vessel wall and from increased temperatures due to blood build-up, the risk of fiber damage, breakage and malfunction is reduced.

The insulating tip 27 and the sheath shaft 7 are permanently attached together at bonding zone 29, as shown in FIG. 3. Although a silicone heat bond is preferably utilized to bond the insulating tip 27 and the shaft 7 together, any suitable standard welding/melting methods may be used to permanently fuse the insulating tip 27 and the sheath shaft 7 together at the bonding zone 29. For example, the distal end of the sheath shaft 7 may be flared outwardly using an RF heating process and then slid over bonding zone 29 of the insulating tip 27. The bonding zone 29 surface can then be heat treated to permanently adhere the sheath shaft 7 material to the insulating tip 27. The bonding zone 29 may then be sanded using techniques well known in the art to achieve a final smooth outer surface. Alternatively, a shrink tubing segment may be placed over bonding zone 29 and heated until a smooth outer profile is achieved.

The length of the bonding zone 29 is approximately 0.080 inches. The length between the front face 43 of the insulating tip 27 and the distal most edge of the insulating tip 27/sheath shaft 7 bonding zone 29 is approximately 0.670 inches. The length between the front face 43 of the insulating tip 27 and the proximal most edge of the bonding zone 29 is approximately 0.750 inches.

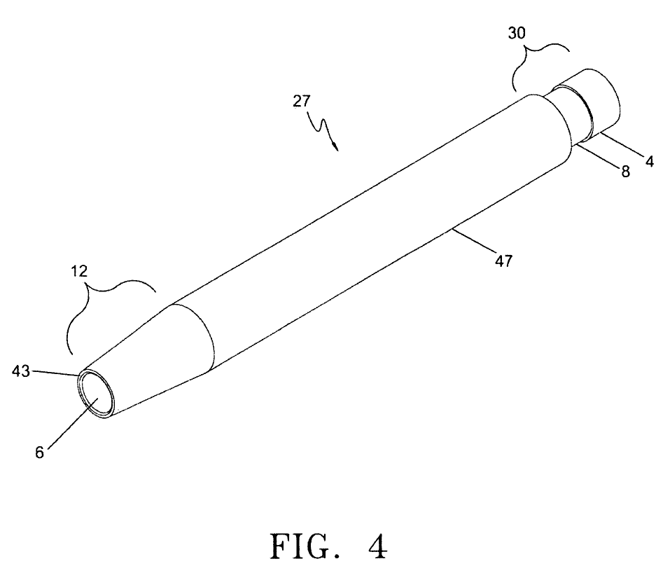

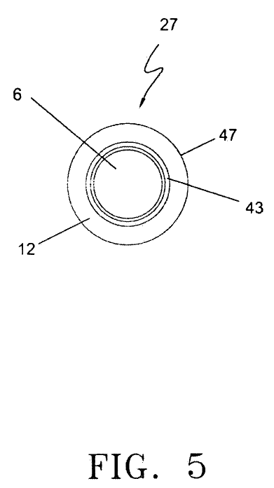

The insulating tip 27 component is illustrated in FIG. 4 and FIG. 5. The insulating tip is approximately 0.750 inches in length. The insulating tip 27 includes a distal tapered tip portion 12, an insulating body 47 and a bonding portion 30. The bonding portion 30 is approximately 0.080 inches in length. A through lumen 6 extends longitudinally from the distal edge 43 to the proximal edge of the bonding portion 30. The bonding portion 30 of the insulating tip 27 is bonded with the sheath shaft 7 at the bonding zone 29. The bonding portion 30 has a ring 4 and a recessed portion 8. The outer diameter of the ring 4 is approximately 0.065 inches. The outer diameter of the recessed portion 8 is approximately 0.060 inches. The ring 4 and the recessed portion 8 are each approximately 0.040 inches in length, respectively. When attached to the sheath shaft 7, the bonding portion 30 is positioned in an interlocking relationship with the distal end of the sheath shaft 7 as shown in FIG. 3. The interlocking relationship provides a supplemental securement mechanism to ensure that the insulating tip 27 and shaft 7 remain attached during pullback of the sheath through the vein.

The insulating tip 27 may be made from a machine-able ceramic, Macor, but finished components could also be molded. Although the tip 27 of the present invention is described herein as being made from a ceramic material, any heat insulative material may be used as the insulating tip 27. The heat insulative material is a material that is both heat-resistive which provides high resistance and structural integrity against high temperature and thermally non-conductive. Such material includes, but not limited to ceramic material, glass, high temperature resistant polymers, carbon, or the like.

The tip 27 of the present invention may contain fluoroscopically visible materials, such as radiopaque fillers, including tungsten or barium sulfate for increased visibility under fluoroscopic imaging. Alternatively or in addition, the tip 27 may have an ultrasonically visible filler such as hollow microspheres which create internal air pockets to enhance the reflective characteristics of the tip 27. With any of these embodiments, the ultrasonic and/or fluoroscopic visibility of sheath tip 27 provides the physician with the option of positioning the sheath tip 27 within the vessel using image guidance. Specifically, the heat insulative sheath tip 27 is more ultrasonically visible than the bare fiber near the light emitting face 37. In one embodiment, the heat insulative sheath tip 27 is also more ultrasonically visible than the shaft 7 of the treatment sheath.

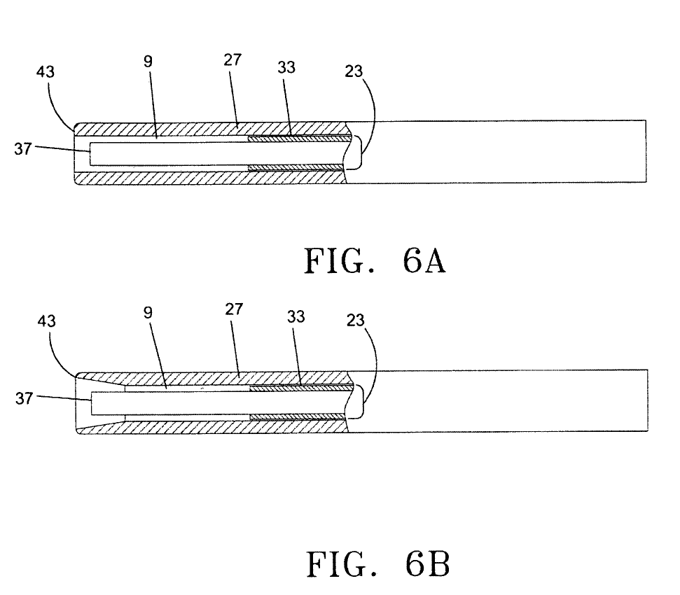

In FIGS. 6A and 6B, the insulating tip 27 is partially cut away to reveal several different embodiments of the distal end of the insulating tip 27 of the present invention. Although the insulating tip 27 is illustrated with a tapered tip in FIGS. 1-5, the distal end may have a straight outer edge, as illustrated in FIG. 6A and/or a straight outer edge with a reverse tapered inner surface whose inner diameter increases in a distal direction, as illustrated in FIG. 6B. In each alternative embodiment, the light emitting face 37 of the optical fiber shaft 23 remains centered inside of the lumen 6 of the insulating tip 27. The face 37 of the fiber shaft 23 also remains protected in a recessed position inside of the insulating tip 27, which completely surrounds the optical fiber shaft 23. In one embodiment of the present invention, the energy delivery portion can be positioned substantially flush with the distal end of the heat insulative tip. In addition to the various insulating tip 27 distal end embodiments illustrated in FIGS. 6A and 6B, and provided that the optical fiber shaft 23 remains protected by the tip 27, one of ordinary skill in the art would recognize that the possibilities are virtually limitless as to what shape or size the insulating sheath tip 27 may be or what materials can be used of the present invention.

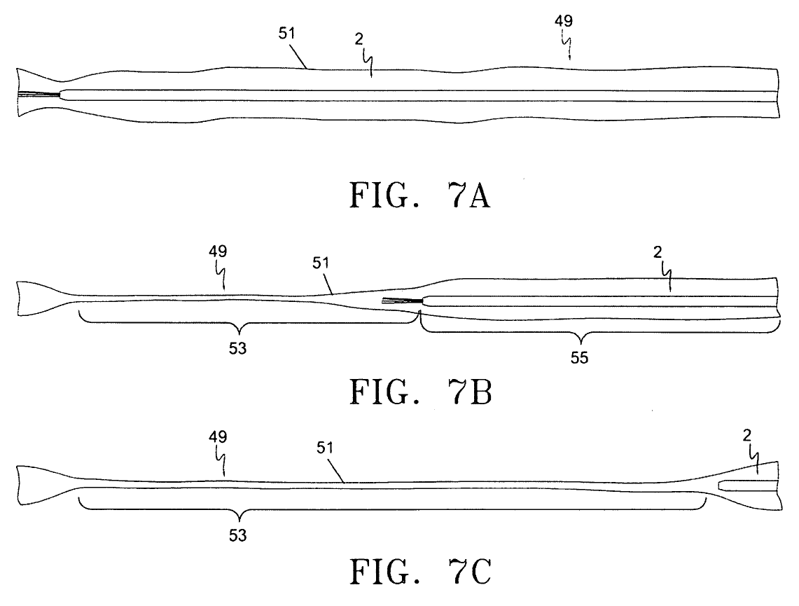

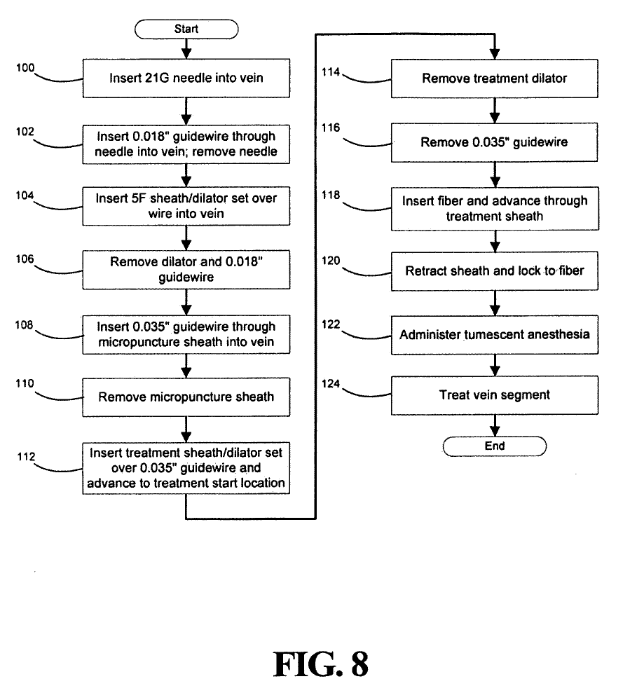

FIGS. 7A, 7B, 7C, and 8 illustrate the procedural steps associated with performing endovenous treatment using the treatment sheath with heat insulative tip and energy delivery device, which is depicted in FIGS. 1-5. To begin the procedure, the target vein is accessed using a standard Seldinger technique well known in the art. Under ultrasonic or fluoroscopic guidance, a small gauge (21G) needle is used to puncture the skin and access the vein (100). A 0.018 inches guidewire is advanced into the vein through the lumen of the needle. The needle is then removed leaving the guidewire in place (102).

A micropuncture sheath/dilator assembly is then introduced into the vein over the guidewire (104). A micropuncture sheath dilator set, also referred to as an introducer set, is a commonly used medical kit, for accessing a vessel through a percutaneous puncture. The micropuncture sheath set includes a short sheath with internal dilator, typically 5-10 cm in length. This length is sufficient to provide a pathway through the skin and overlying tissue into the vessel, but not long enough to reach distal treatment sites. Once the vein has been access using the micropuncture sheath/dilator set, the dilator and 0.018 inches guidewire are removed (106), leaving only the micropuncture introducer sheath in place within the vein (106). A 0.035 inches guidewire is then introduced through the introducer sheath into the vein. The guidewire is advanced through the vein until its tip is positioned near the sapheno-femoral junction or other starting location within the vein (108).

After removing the micropuncture sheath (110), a treatment sheath/dilator set is introduced into the vein and advanced over the 0.035 inches guidewire and advanced to 1 to 2 centimeters below the point of reflux, typically until the tip of the treatment sheath is positioned near the sapheno-femoral junction or other reflux point (112). Unlike the micropuncture introducer sheath, the treatment sheath is of sufficient length to reach the location within the vessel where the laser treatment will begin, typically the sapheno-femoral junction. Typical treatment sheath lengths are 45 and 65 cm. Positioning of the treatment sheath 2 is confirmed using either ultrasound or fluoroscopic imaging. The insulative tip 27 is designed to be clearly visible under either ultrasound or fluoroscopy. Once the treatment sheath/dilator set is correctly positioned within the vessel, the dilator component and guidewire are removed from the treatment sheath (114, 116).

The energy delivery device 10 is then inserted into the treatment sheath lumen and advanced until the energy delivery portion is surrounded by the heat insulative tip of the treatment sheath (118). If the fiber assembly has a connector lock 60 as shown in FIG. 1B and as described in U.S. Pat. No. 7,033,347, also incorporated herein by reference, the treatment sheath and fiber assembly are locked together by the two luer type connectors 29, 60 to maintain the position of the energy delivery portion during pullback (120). Locking the two components together automatically positions the energy delivery portion relative to the sheath insulative tip. Correct positioning of the distal end of the insulative tip 27 of sheath 2 approximately 1-2 centimeters below the sapheno-femoral junction or other reflux point is once again confirmed using ultrasound or fluoroscopy. Unlike prior art devices, the physician is not required to visually confirm positioning of the fiber tip 37, since it is automatically aligned in the desired position within the sheath 2.

The physician may optionally administer tumescent anesthesia along the length of the vein (122). Tumescent fluid may be injected into the peri-venous anatomical sheath surrounding the vein and/or is injected into the tissue adjacent to the vein, in an amount sufficient to provide the desired anesthetic effect and to thermally insulate the treated vein from adjacent structures including nerves and skin. Once the vein has been sufficiently anesthetized, laser energy or the like is applied to the interior of the diseased vein segment 49. The laser generator (not shown) is turned on, and as illustrated in FIG. 7A, the laser light is emitted through the emitting face of the optical fiber. While the laser light is emitting laser light through the emitting face, the treatment sheath/fiber assembly is withdrawn through the vessel at a pre-determined rate, typically 2-3 millimeters per second (124). The laser energy that is used to perform the endovenous thermal therapy may be of a wavelength of 980 nm, but other wavelengths may be used as well. The laser energy travels along the optical fiber shaft through the energy-emitting face of the fiber and into the vein lumen, where the laser energy is absorbed by the blood present in the vessel and, in turn, is converted to thermal energy to substantially uniformly heat the vein wall along a 360 degree circumference, thus damaging the vein wall tissue, causing cell necrosis and ultimately causing collapse/occlusion of the vessel.

The physician manually controls the rate at which the sheath 2 and optical fiber 10 are withdrawn. As an example, it takes approximately 3 minutes to treat a 45 centimeter vein segment 49, and it requires a pullback rate of about one centimeter every four seconds. The laser energy produces localized thermal injury to the endothelium and vein wall 51 causing occlusion of the vein. The laser energy travels down the optical fiber shaft 23 through the energy-emitting face 37 of the optical fiber shaft 23 and into the vein lumen, where thermal energy contacts the blood, causing hot bubbles of gas to be created in the bloodstream. The gas bubbles expand to contact the vein wall 51, along a 360 degree circumference, thus damaging vein wall 51 tissue, causing cell necrosis, and ultimately causing collapse of the vessel.

Misdirected delivery of laser energy may result in vessel wall perforations where heat is concentrated and incomplete tissue necrosis where insufficient thermal energy is delivered. The endovascular treatment device of the present invention with a optical fiber shaft 23 that is protected by an insulating tip 27 avoids these problems by preventing inadvertent contact between the face 37 of the optical fiber shaft 23 and the vessel's inner wall 51 as the sheath 2 and optical fiber 10 are withdrawn through the vessel. The insulating tip 27 extends over and is spaced radially away from the light emitting face 37 of the optical fiber shaft 23 to prevent even inadvertent vessel wall contact. Although thin, the insulating tip 27 provides the necessary barrier between the vessel wall 51 and the optical fiber face 37 to prevent unequal laser energy delivery and fragmentation of the optical fiber shaft 23.

As illustrated in FIGS. 7B and 7C, section 53 of the diseased vein segment 49 has been treated with laser energy and is reduced in diameter. Section 55 of the diseased vein segment 49 has not been thermally damaged by the steam bubbles created by the emission of laser energy into the blood and thus remains open and dilated. After the entire vein segment 49 has been treated, the thermally damaged vessel will eventually become occluded and can no longer support blood flow.

The procedure for treating the varicose vein is considered to be complete when the desired length of the target vein has been exposed to laser energy. Normally, the laser generator is turned off when the face 37 of the optical fiber shaft 23 is approximately 3 centimeters from the access site. The physician can monitor the location of the face 37 relative to the puncture site in two different ways. Once the physician has been alerted to the proximity of the distal end of the insulating tip 27 at the access site by optional depth markers on the sheath 2, the physician continues to pull back the sheath 2 and optical fiber 10 until the bonding zone 29 appears at the access site indicating that the light emitting face 37 of the optical fiber 10 will be approximately 3 centimeters below the skin opening. At this point, the generator is turned off and the sheath 2 and optical fiber 10 can then be removed from the body.

The invention disclosed herein has numerous advantages over prior art treatment devices and methods. The endovascular sheath with its heat insulative tip and method of the present invention provides increased integrity of the treatment sheath by shielding the heat caused by laser energy from traveling upstream and burning the treatment sheath. The present device also provides optimized visibility under ultrasonic imaging modalities. This enhanced visibility of the insulating tip 27 of the sheath 2 leads to increased accuracy during final positioning of the device near the sapheno-femoral junction.

Finally, the insulating tip 27 of the present invention protects the delicate optical fiber shaft 23 during the endovenous laser treatment therapy, which prevents the optical fiber tip 37 from inadvertently contacting the vessel wall, thereby avoiding the problems described above, such as incomplete treatment, vessel perforations, or fragmentation, thereby further enhancing the endovenous laser therapy treatment.

Also veins other than the great saphenous vein can be treated using the method described herein.

The above disclosure is intended to be illustrative and not exhaustive. This description will suggest many modifications, variations, and alternatives may be made by ordinary skill in this art without departing from the scope of the invention. Those familiar with the art may recognize other equivalents to the specific embodiments described herein. Accordingly, the scope of the invention is not limited to the foregoing specification.

What is claimed is:

1. An endovascular thermal treatment device for causing closure of a blood vessel comprising:
an energy delivery device having an energy delivery portion at its distal end; and
a treatment sheath having a shaft adapted to receive the energy delivery device and a heat insulative tip arranged near a distal end of the shaft.

2. The endovascular thermal treatment device of claim 1, wherein the energy delivery device is a laser energy delivery device.

3. The endovascular thermal treatment device of claim 1, wherein the energy delivery device is an optical fiber having a light emitting face.

4. The endovascular thermal treatment device of claim 1, wherein the heat insulative tip has a higher ultrasonic visibility than the energy delivery portion.

5. The endovascular thermal treatment device of claim 1, wherein the heat insulative tip includes ceramic material.

6. The endovascular thermal treatment device of claim 1, wherein the heat insulative tip includes high temperature resistant polymer.

7. The endovascular thermal treatment device of claim 1, wherein the heat insulative tip includes glass material.

8. The endovascular thermal treatment device of claim 1, wherein the heat insulative tip includes carbon material.

9. The endovascular thermal treatment device of claim 1, wherein the heat insulative tip is tapered.

10. The endovascular thermal treatment device of claim 9, wherein a distal portion of the heat insulative tip is tapered.

11. The endovascular thermal treatment device of claim 1, wherein the heat insulative tip has a reverse tapered inner surface whose inner diameter increases in a distal direction.

12. The endovascular thermal treatment device of claim 1, wherein:
the treatment sheath has at its proximal end a first connector; and
the energy delivery device has a second connector arranged at a predetermined distance from the energy delivery portion such that when the first and second connectors are locked together, the distal end of the energy delivery portion is positioned near the distal end of the heat insulative tip and inside the heat insulative tip.

13. The endovascular thermal treatment device of claim 12, wherein:
the energy delivery device is an optical fiber having a light emitting face; and
when the first and second connectors are locked together, the light emitting face is positioned substantially flush with the distal end of the heat insulative tip.

14. The endovascular thermal treatment device of claim 12, wherein:
the energy delivery device is an optical fiber having a light emitting face; and
when the first and second connectors are locked together, the light emitting face is recessed from the distal end of the heat insulative tip by a predetermined distance.

15. The endovascular thermal treatment device of claim 1, wherein:
the energy delivery device is an optical fiber having a jacket that surrounds a fiber core and the energy delivery portion that includes a distal fiber portion whose jacket has been stripped; and
the inner diameter of the heat insulative tip is smaller than the inner diameter of the shaft of the treatment sheath;
the outer diameter of the jacket is about equal to the inner diameter of the heat insulative tip so as to center the energy delivery portion within the heat insulative tip.

16. The endovascular thermal treatment device according to claim 1, further comprising a plurality of spaced markers disposed along a wall of the treatment sheath.

17. The endovascular thermal treatment device according to claim 1, wherein the heat insulative tip is permanently attached to the distal end of the shaft.

18. An endovascular laser treatment device for causing closure of a blood vessel comprising:
an optical fiber having a light emitting face at its distal end; and
a treatment sheath having a shaft adapted to receive the optical fiber and a heat insulative tip arranged near a distal end of the shaft.

19. The endovascular laser treatment device of claim 1, wherein the heat insulative tip has a higher ultrasonic visibility than the optical fiber near the light emitting face.

20. The endovascular laser treatment device of claim 1, wherein the heat insulative tip includes ceramic material.

21. The endovascular laser treatment device of claim 1, wherein the heat insulative tip includes high temperature resistant polymer, glass material, carbon material or a combination thereof.

22. The endovascular laser treatment device of claim 1, wherein the heat insulative tip is tapered.

23. The endovascular laser treatment device of claim 1, wherein the heat insulative tip has a reverse tapered inner surface whose inner diameter increases in a distal direction.

24. The endovascular laser treatment device of claim 1, wherein:
the treatment sheath has at its proximal end a first connector; and
the optical fiber has a second connector arranged at a predetermined distance from the light emitting face such that when the first and second connectors are locked together, the distal end of the light emitting face is positioned near the distal end of the heat insulative tip and inside the heat insulative tip.

25. The endovascular laser treatment device of claim 24, wherein when the first and second connectors are locked together, the light emitting face is positioned substantially flush with the distal end of the heat insulative tip.

26. The endovascular laser treatment device of claim 24, wherein when the first and second connectors are locked together, the light emitting face is recessed from the distal end of the heat insulative tip by a predetermined distance.

27. An endovascular treatment method for causing closure of a blood vessel comprising:
inserting into a blood vessel a treatment sheath having a shaft and a heat insulative tip attached to a distal end of the shaft;
inserting through the treatment sheath an energy delivery device having an energy delivery portion at its distal end; and
applying thermal energy through the energy delivery portion while longitudinally moving the inserted optical fiber and the treatment sheath together, the heat insulative tip positioning the energy delivery portion away from an inner wall of the blood vessel.

28. The endovascular treatment method of claim 1, wherein the energy delivery device is an optical fiber having a light emitting face at its distal end.

29. The endovascular treatment method of claim 1, wherein the heat insulative tip has a higher ultrasonic visibility than the energy delivery portion.

30. The endovascular treatment method of claim 1, wherein the heat insulative tip includes ceramic material.

31. The endovascular treatment method of claim 1, before the step of applying thermal energy, further comprising locking together the treatment sheath and the energy delivery device such that the distal end of the energy delivery portion is positioned near the distal end of the heat insulative tip and inside the heat insulative tip.

32. The endovascular treatment method of claim 1, wherein the heat insulative tip has a higher ultrasonic visibility than the energy delivery portion, and the method further comprises, prior to the step of applying thermal energy, positioning the energy delivery portion within the vessel using ultrasonic guidance of the heat insulative tip.

33. The endovascular treatment method of claim 1, wherein the step of applying thermal energy through the energy delivery portion includes applying thermal energy while withdrawing the inserted energy delivery device and the treatment sheath together.
